SEQUENCE LISTING (1 sequence)

SEQ ID NO: 1 - DNA, 46 bp, Enterobacteria phage T7
  misc_feature 45, 46: n = A,T,C or G
aattaatacgactcactatagggagattttttttttttttttttnn

(19) United States
(12) Patent Application Publication
(10) Pub. No.: US 2005/0136411 A1
(43) Pub. Date: Jun. 23, 2005

(54) Methods and compositions for linear mRNA amplification from small RNA samples

(76) Inventors: Patrick J. Collins, San Francisco, CA (US); Khanh H.N. Nguyen, San Jose, CA (US); Diane D. Ilsley, San Jose, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/744,434

(22) Filed: Dec. 22, 2003

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

Methods and compositions for linearly amplifying mRNA from small RNA samples to produce antisense RNA are provided In the subject methods, mRNA is converted to a transcription template product by using a promoter primer, where the resultant product at least includes a first strand cDNA template domain and a double-stranded RNA polymerase promoter. A feature of the subject methods is that the ratio of the promoter-primer to the input RNA is chosen to provide for a high yield of selective RNA product. Additional optional features include the use of a thermostabilizing agent, e.g., raffinose, trehalose, etc., in the reverse transcription step and/or the use of a polyalkylene oxide, e.g., a PEG, in the in vitro transcription step. Also provided are compositions and kits that find use in practicing the subject methods. The subject invention finds use a variety of different applications in which the preparation of linearly amplified amounts of cRNA is desired.

### TECHNICALl FIELD

The technical field of this invention is the amplification of nucleic acids.

### BACKGROUND OF THE INVENTION

The characterization of cellular gene expression finds application in a variety of disciplines, such as in the analysis of differential expression between different tissue types, different stages of cellular growth or between normal and diseased states. Fundamental to differential expression analysis is the detection of different mRNA species in a test population, and the quantitative determination of different mRNA levels in that test population. However, the detection of rare mRNA species is often complicated by one or more of the following factors: cell heterogeneity, paucity of material, or the limits of detection of the assay method. Thus, methods which amplify heterogeneous populations of mRNA that do not introduce significant changes in the relative amounts of different mRNA species facilitate this technology.

A number of methods for the amplification of nucleic acids have been described. Such methods include the “polymerase chain reaction” (PCR) (Mullis et al., U.S. Pat. No. 4,683,195), and a number of transcription-based amplification methods (Malek et al., U.S. Pat. No. 5,130,238; Kacian and Fultz, U.S. Pat. No. 5,399,491; Burg et al., U.S. Pat. No. 5,437,990). Each of these methods uses primer-dependent nucleic acid synthesis to generate a DNA or RNA product, which serves as a template for subsequent rounds of primer-dependent nucleic acid synthesis. Each process uses (at least) two primer sequences complementary to different strands of a desired nucleic acid sequence and results in an exponential increase in the number of copies of the target sequence. These amplification methods can provide enormous amplification (up to billion-fold). However, these methods have limitations that make them not amenable for gene expression monitoring applications. First, each process results in the specific amplification of only the sequences that are bounded by the primer binding sites. Second, exponential amplification can introduce significant changes in the relative amounts of specific target species—small differences in the yields of specific products (for example, due to differences in primer binding efficiencies or enzyme processivity) become amplified with every subsequent round of synthesis.

Amplification methods that utilize a single primer are amenable to the amplification of heterogeneous mRNA populations. The vast majority of mRNAs carry a homopolymer of 20-250 adenosine residues on their 3′ ends (the poly-A tail), and the use of poly-dT primers for cDNA synthesis is a fundamental tool of molecular biology. “Single-primer amplification” protocols have been reported (see e.g. Kacian et al., U.S. Pat. No. 5,554,516; Van Gelder et al., U.S. Pat. No. 5,716,785). The methods reported in these patents utilize a single primer containing an RNA polymerase promoter sequence and a sequence complementary to the 3′-end of the desired nucleic acid target sequence(s) (“promoter-primer”). In both methods, the promoter-primer is added under conditions where it hybridizes to the target sequence(s) and is converted to a substrate for RNA polymerase. In both methods, the substrate intermediate is recognized by RNA polymerase, which produces multiple copies of RNA complementary to the target sequence(s) (“antisense RNA”). Each method uses, or could be adapted to use, a primer containing poly-dt for amplification of heterogeneous mRNA populations.

Amplification methods that proceed linearly during the course of the amplification reaction are less likely to introduce bias in the relative levels of different mRNAs than those that proceed exponentially. In the method described in U.S. Pat. No. 5,554,516, the amplification reaction contains a nucleic acid target sequence, a promoter-primer, an RNA polymerase, a reverse transcriptase, and reagent and buffer conditions sufficient to allow amplification. The amplification proceeds in a single tube under conditions of constant temperature and ionic strength. Under these conditions, the antisense RNA products of the reaction can serve as substrates for further amplification by non-specific priming and extension by the RNA-dependent DNA polymerase activity of reverse transcriptase. As such, the amplification described in U.S. Pat. No. 5,554,516 proceeds exponentially. In contrast, in specific examples described in U.S. Pat. No. 5,716,785, cDNA synthesis and transcription occur in separation reactions separated by phenol/chloroform extraction and ethanol precipitation (or dialysis), which may incidentally allow for the amplification to proceed linearly since the RNA products cannot serve as substrates for further amplification.

The method described in U.S. Pat. No. 5,716,785 has been used to amplify cellular mRNA for gene expression monitoring (for example, R. N. Van Gelder et al. (1990), Proc. Natl. Acad. Sci. USA 87, 1663; D. J. Lockhart et al. (1996), Nature Biotechnol. 14, 1675). However, this procedure is not readily amenable to high throughput processing. In preferred embodiments of the method described in U.S. Pat. No. 5,716,785, poly-A mRNA is primed with a promoter-primer containing poly-dT and converted into double-stranded cDNA using a method described by Gubler and Hoffman (U. Gubler and B. J. Hoffman (1983), Gene 25, 263-269) and popularized by commercially available kits for cDNA synthesis. Using this method for cDNA synthesis, first strand synthesis is performed using reverse transcriptase and second strand cDNA is synthesized using RNaseH and DNA polymerase 1. After phenol/chloroform extraction and dialysis, double-stranded cDNA is transcribed by RNA polymerase to yield antisense RNA product.

One problem with the above-described methods is that purified mRNA is used as the input RNA Isolation of mRNA directly from cells and total RNA can result in significant losses of low abundant messages and a portion of the overall population. In addition, the process is time consuming and the yields are low, especially from a low number of cells. Polyadenylated RNA can be isolated directly from lysed cells, but guanidine salts used to inhibit RNAses are highly disruptive of binding and can interfere with mRNA binding to an oligodT support.

Accordingly, there is interest in the development of improved methods of antisense RNA amplification. Of particular interest would be the development of a protocol which allowed for the use of total RNA as opposed to isolated mRNA as the input RNA.

Relevant Literature

United States Patents of interest include: U.S. Pat. Nos. 6,132,997; 5,932,451; 5,716,785; 5,554,516; 5,545,522; 5,437,990; 5,130,238; and 5,514,545. Antisense RNA synthesis is also discussed in Phillips and Eberwine (1996), Methods: A companion to Methods in Enzymol. 10, 283; Eberwine et al. (1992), Proc., Natl., Acad. Sci. USA 89, 3010; Eberwine (1996), Biotechniques 20, 584; and Eberwine et al. (1992), Methods in Enzymol. 216, 80.

### SUMMARY OF THE INVENTION

Methods and compositions for linearly amplifying mRNA from small initial RNA samples to produce antisense RNA are provided. In the subject methods, mRNA is converted to a transcription template product by using a promoter primer, where the resultant product at least includes a first strand cDNA template domain and a double-stranded RNA polymerase promoter. In certain representative embodiments, the transcription template product is produced by converting mRNA to double-stranded cDNA in a reverse transcription step using a promoter-primer having a primer site linked to a promoter sequence. The resultant transcription template product is then transcribed into cRNA. A feature of the subject methods is that the ratio of the promoter-primer to the input RNA is chosen to provide for a high yield of selective RNA product. Additional optional features include the use of a thermostabilizing agent, e.g., raffinose, trehalose, etc., in the reverse transcription step and/or the use of a polyalkylene oxide, e.g., a PEG, in the in vitro transcription step. Also provided are compositions and kits that find use in practicing the subject methods. The subject invention finds use a variety of different applications in which the preparation of linearly amplified amounts of cRNA is desired.

### BRIEF DESCRIPTION OF THE FIGURES

FIG. 1 shows an exemplary substrate carrying an array, such as may be used in the devices of the subject invention.

FIG. 2 shows an enlarged view of a portion of FIG. 1 showing spots or features.

FIG. 3 is an enlarged view of a portion of the substrate of FIG. 1.

FIGS. 4A and 4B show the linear correlation of the log ratio of amplified cRNA targets as a function of sample input.

FIG. 5 provides a titration curve of T7 promoter primer from 0.05 μM to 1.5 μM in the presence and absence of 100 ng of HeLa total RNA.

FIG. 6 provides a comparison of the effect of two additives, i.e., raffinose or betaine, to the RT reaction with 100 ng of HeLa total RNA.

FIGS. 7A to 8D provide graphical results of a Standard protocol described in the Experimental Section on various amounts of input RNA.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

A “biopolymer” is a polymeric biomolecule of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups.

A “biomonomer” references a single unit, which can be linked with the same or other biomonomers to form a biopolymer (e.g., a single amino acid or nucleotide with two linking groups, one or both of which may have removable protecting groups).

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

With respect to methods in which premade probes are immobilized on a substrate surface, immobilization of the probe to a suitable substrate may be performed using conventional techniques. See, e.g., Letsinger et al. (1975) Nucl. Acids Res. 2: 773-786; Pease, A. C. et al., Proc. Nat. Acad. Sci. USA, 1994, 91: 5022-5026. The surface of a substrate may be treated with an organosilane coupling agent to functionalize the surface. One exemplary organosilane coupling agent is represented by the formula RₙSiY₍₄₋ₙ₎ wherein: Y represents a hydrolyzable group, e.g., alkoxy, typically lower alkoxy, acyloxy, lower acyloxy, amine, halogen, typically chlorine, or the like; R represents a nonhydrolyzable organic radical that possesses a functionality which enables the coupling agent to bond with organic resins and polymers; and n is 1, 2 or 3, usually 1. One example of such an organosilane coupling agent is 3-glycidoxypropyltrimethoxysilane (“GOPS”), the coupling chemistry of which is well-known in the art. See, e.g., Arkins, “Silane Coupling Agent Chemistry,” Petrarch Systems Register and Review, Eds. Anderson et al. (1987). Other examples of organosilane coupling agents are (γ-aminopropyl)triethoxysilane and (γ-aminopropyl)trimethoxysilane. Still other suitable coupling agents are well known to those skilled in the art. Thus, once the organosilane coupling agent has been covalently attached to the support surface, the agent may be derivatized, if necessary, to provide for surface functional groups. In this manner, support surfaces may be coated with functional groups such as amino, carboxyl, hydroxyl, epoxy, aldehyde and the like.

Use of the above-functionalized coatings on a solid support provides a means for selectively attaching probes to the support. For example, an oligonucleotide probe formed as described above may be provided with a 5′-terminal amino group that can be reacted to form an amide bond with a surface carboxyl using carbodiimide coupling agents. 5′ attachment of the oligonucleotide may also be effected using surface hydroxyl groups activated with cyanogen bromide to react with 5′-terminal amino groups. 3′-terminal attachment of an oligonucleotide probe may be effected using, for example, a hydroxyl or protected hydroxyl surface functionality.

In situ prepared ligand arrays, e.g., nucleic acid arrays, may be characterized by having surface properties of the substrate that differ significantly between the feature and inter-feature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water. “Contact angle” of a liquid with a surface is the acute angle measured between the edge of a drop of liquid on that surface and the surface. Contact angle measurements are well known and can be obtained by various instruments such as an FTA200 available from First Ten Angstroms, Portsmouth, Va., U.S.A. Surfaces which are more hydrophobic (which have a lower surface energy) will have higher contact angles with water or aqueous liquids than surfaces which are less hydrophobic (and therefore a higher surface energy) (for example, a hydrophobic surface may have a water drop contact angle of more than 50 degrees, or even more than 90 degrees). The contact angle of an array (sometimes referenced as the “average contact angle” or “effective contact angle”) is the average contact angle of the features of that array and the inter-feature areas. Contact angles are measured with water unless otherwise indicated.

In certain embodiments, high surface energy regions, e.g., features, may have contact angles that are less than 45 degrees, less than 20 degrees (or less than 15, 10, or 5 degrees), while low surface energy, e.g., inter-feature, areas may have contact angles greater than 80 degrees (or even greater than 90, 95, 100, 105, 110, 115, 120 or 130 degrees).

Also, instead of drop deposition methods, light directed fabrication methods may be used, as are known in the art. Inter-feature areas need not be present particularly when the arrays are made by light directed synthesis protocols.

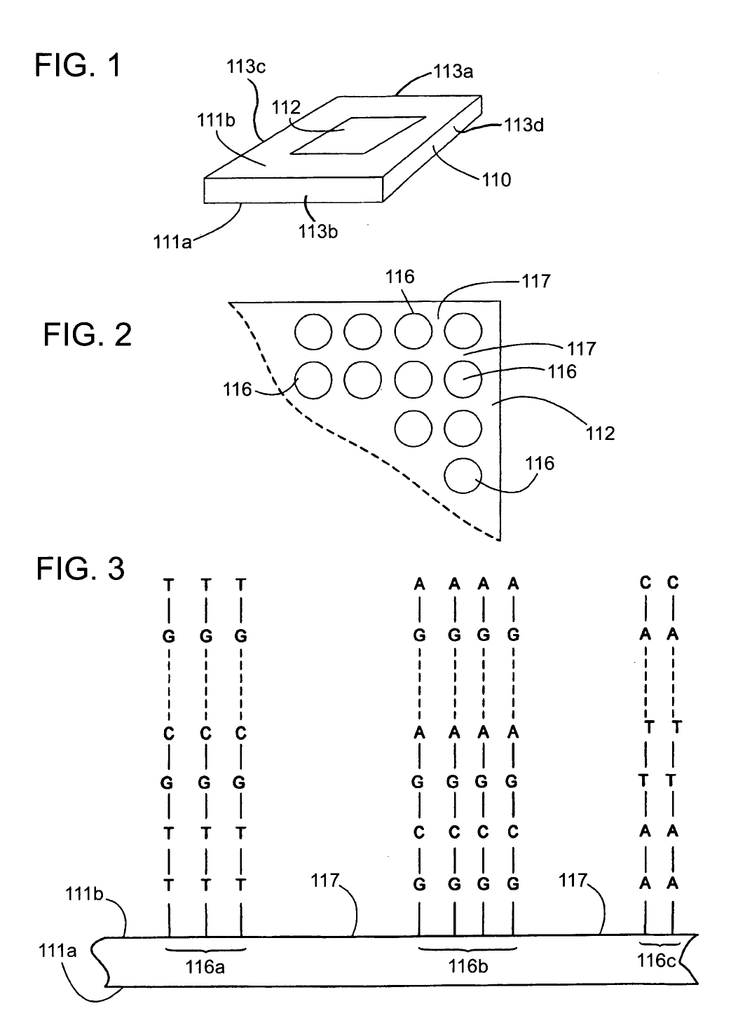

An exemplary array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure, e.g., a bottom surface or a cover that does not readily bend without breakage, i.e., the structure is not flexible.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “sample” as used herein relates to a material or mixture of materials, typically, although not necessarily, in fluid form, containing one or more components of interest.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DESCRIPTION OF THE SPECIFIC EMBODIMENTS

Methods and compositions for linearly amplifying mRNA from small RNA samples to produce antisense RNA are provided. In the subject methods, mRNA is converted to a transcription template product by using a promoter primer, where the resultant product at least includes a first strand cDNA template domain and a double-stranded RNA polymerase promoter. In certain representative embodiments, the transcription template product is produced by converting mRNA to double-stranded cDNA in a reverse transcription step using a promoter-primer having a primer site linked to a promoter sequence. The resultant transcription template product is then transcribed into cRNA, which is also antisense RNA with respect to the corresponding initial mRNA. A feature of the subject methods is that the ratio of the promoter-primer to the input RNA is chosen to provide for a high yield of selective RNA product. Additional optional features include the use of a thermostabilizing agent, e.g., raffinose, trehalose, etc., in the reverse transcription step and/or the use of a polyalkylene oxide, e.g., a PEG, in the in vitro transcription step. Also provided are compositions and kits that find use in practicing the subject methods. The subject invention finds use a variety of different applications in which the preparation of linearly amplified amounts of cRNA is desired.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications which might be used in connection with the presently described invention.

As summarized above, the present invention provides methods of preparing amplified amounts of cRNA from mRNA using total RNA as the mRNA source, as well as kits for use in practicing the subject methods. In further describing the present invention, the subject methods are discussed first in greater detail, followed by a review of representative kits for use in practicing the subject methods.

Methods

The subject invention provides methods for linearly amplifying mRNA into antisense RNA. As such, the subject invention provides methods of producing amplified amounts of antisense RNA from an initial amount of mRNA. By amplified amounts is meant that for each initial mRNA, multiple corresponding antisense RNAs are produced, where the term antisense RNA is defined here as ribonucleic acid complementary to the initial mRNA. By corresponding is meant that the antisense RNA shares a substantial amount of sequence identity with the sequence complementary to the mRNA (i.e. the complement of the initial mRNA), where substantial amount means at least 95%, such as at least 98% and including at least 99%, where sequence identity is determined using the BLAST algorithm, as described in Altschul et al. (1990), J. Mol. Biol. 215: 403410 (using the published default setting, i.e. parameters w=4, t=17). Generally, the number of corresponding antisense RNA molecules produced for each initial mRNA during the subject linear amplification methods will be at least about 10, such as at least about 50 and including at least about 100, where the number may be as great as about 600 or greater, but often does not exceed about 10000.

In the first step of the subject methods (i.e., the reverse transcription step), an initial mRNA sample is converted to a transcription template product by using a promoter primer, where the resultant transcription template product at least includes a first strand cDNA template domain and a double-stranded RNA polymerase promoter. In the broadest sense, the transcription template product need only include a single-stranded first strand cDNA domain and a double stranded RNA polymerase promoter domain. Accordingly, in certain embodiments, a promoter primer that includes a hairpin promoter domain (which makes it double stranded) and a primer domain may be employed in first strand cDNA synthesis to produce a transcription template product that includes a single-stranded first strand cDNA domain and a double stranded RNA polymerase promoter domain. Alternatively, instead of the using a primer with a hairpin promoter domain, one could using a promoter primer to synthesize first strand cDNA, and then hybridize a complementary sequence to the promoter domain to produce the requisite double stranded promoter domain.

In certain representative embodiments, the transcription template product is produced by converting mRNA to double-stranded cDNA in a reverse transcription step using a promoter-primer having a primer site linked to a promoter sequence. In these representative embodiments, the initial mRNA is subjected to a series of enzymatic reactions under conditions sufficient to ultimately produce double-stranded DNA for each initial mRNA in the sample that is amplified. During this first step, an RNA polymerase-promoter region is incorporated into the resultant product, which region is employed in the second step of the subject methods, i.e. the transcription step described in greater detail infra.

A feature in many embodiments of the subject methods is that the initial RNA sample that is employed is a total RNA sample. As such, in these embodiments, a total RNA preparation is employed as the source of mRNA. The initial RNA sample, e.g., total RNA sample, will typically be derived from a physiological source. The physiological source may be derived from a variety of eukaryotic sources, with physiological sources of interest including sources derived from single-celled organisms such as yeast and multicellular organisms, including plants and animals, particularly mammals, where the physiological sources from multicellular organisms may be derived from particular organs or tissues of the multicellular organism, or from isolated cells derived therefrom. In obtaining the total RNA preparation from the physiological source from which it is derived, any convenient protocol for isolation of total RNA from the initial physiological source may be employed. Methods of isolating RNA from cells, tissues, organs or whole organisms are known to those of skill in the art and include those described in Maniatis et al. (1989), Molecular Cloning: A Laboratory Manual 2d Ed. (Cold Spring Harbor Press).

Depending on the nature of the primer employed during first strand synthesis, as described in greater detail below, the subject methods can be used to produce amplified amounts of antisense RNA corresponding to substantially all of the mRNA present in the initial sample, or to a proportion or fraction of the total number of distinct mRNAs present in the initial sample. By substantially all of the mRNA present in the sample is meant more than 90%, usually more than 95%, where that portion not amplified is solely the result of inefficiencies of the reaction or the enzyme and not intentionally excluded from amplification.

The promoter-primer employed in the amplification reaction includes: (a) a poly-dT region for hybridization to the poly-A tail of the mRNA; and (b) an RNA polymerase promoter region 5′ of the -poly-dT region that is in an orientation capable of directing transcription of antisense RNA. In certain embodiments, the primer will be a “lock-dock” primer, in which immediately 3′ of the poly-dT region is either a “G”, “C”, or “A” such that the primer has the configuration of 3′-XTTTTTTT . . . 5′, where X is “G”, “C”, or “A”. The poly-dT region is sufficiently long to provide for efficient hybridization to the poly-A tail, where the region typically ranges in length from 10-50 nucleotides in length, usually 10-25 nucleotides in length, and more usually from 14 to 20 nucleotides in length.

A number of RNA polymerase promoters may be used for the promoter region of the first strand cDNA primer, i.e. the promoter-primer. Suitable promoter regions will be capable of initiating transcription from an operationally linked DNA sequence in the presence of ribonucleotides and an RNA polymerase under suitable conditions. The promoter will be linked in an orientation to permit transcription of antisense RNA. A linker oligonucleotide between the promoter and the DNA may be present, and if, present, will typically comprise between about 5 and 20 bases, but may be smaller or larger as desired. The promoter region will usually comprise between about 15 and 250 nucleotides, preferably between about 17 and 60 nucleotides, from a naturally occurring RNA polymerase promoter or a consensus promoter region, as described in Alberts et al. (1989) in Molecular Biology of the Cell, 2d Ed. (Garland Publishing, Inc.). In general, prokaryotic promoters are preferred over eukaryotic promoters, and phage or virus promoters most preferred. As used herein, the term “operably linked” refers to a functional linkage between the affecting sequence (typically a promoter) and the controlled sequence (the mRNA binding site). The promoter regions that find use are regions where RNA polymerase binds tightly to the DNA and contain the start site and signal for RNA synthesis to begin. A wide variety of promoters are known and many are very well characterized. Representation promoter regions of particular interest include T7, T3 and SP6 as described in Chamberlin and Ryan, The Enzymes (ed. P. Boyer, Academic Press, New York) (1982) pp 87-108. As indicated above, in certain embodiments the promoter domain may be a hairpin domain which can serve to promote transcription of a single stranded cDNA domain.

Where one wishes to amplify only a portion of the mRNA species in the sample, one may optionally provide for a short arbitrary sequence 3′ of the poly-dT region, where the short arbitrary sequence will generally be less than 5 nucleotides in length and usually less than 2 nucleotides in length, where the dNTP immediately adjacent to the poly-dT region will not be a T residue and usually the sequence will comprise no T residue. Such short 3′ arbitrary sequences are described in Ling and Pardee (1992), Science 257, 967.

The promoter-primer described above and throughout this specification may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al. (1981), Tetrahedron Letters 22, 1859. One method for synthesizing oligonucleotides on a modified solid support is described in U.S. Pat. No. 4,458,066. It is also possible to use a primer that has been isolated from a biological source (such as a restriction endonuclease digest). The primers herein are selected to be “substantially” complementary to each specific sequence to be amplified, i.e.; the primers should be sufficiently complementary to hybridize to their respective targets. Therefore, the primer sequence need not reflect the exact sequence of the target, and can, in fact be “degenerate.” Non-complementary bases or longer sequences can be interspersed into the primer, provided that the primer sequence has sufficient complementarity with the sequence of the target to be amplified to permit hybridization and extension.

In the first step of the subject methods, i.e., the reverse transcription step, the oligonucleotide promoter-primer is hybridized with a sufficient amount of an initial total RNA (containing the mRNA to be amplified) sample (as described above) and the primer-mRNA hybrid is converted to a transcription template product that may include a single-stranded cDNA domain and a double stranded promoter domain, or be a double-stranded cDNA product, where the product is recognized by an RNA polymerase. The amount of initial total RNA sample that is employed may vary, and may be as low as 1000 ng or lower, e.g., about 500 ng or lower, about 400 ng or lower, about 200 ng or lower, including about 100 ng or lower, including about 50 ng or lower, e.g., about 20 ng or lower etc., where the amount in certain representative embodiments ranges from about 10 ng to about 1000 ng, usually from about 10 ng to about 500 ng, and the amount of total RNA sample employed in certain embodiments does not exceed about 500 ng, and may not exceed about 100 ng.

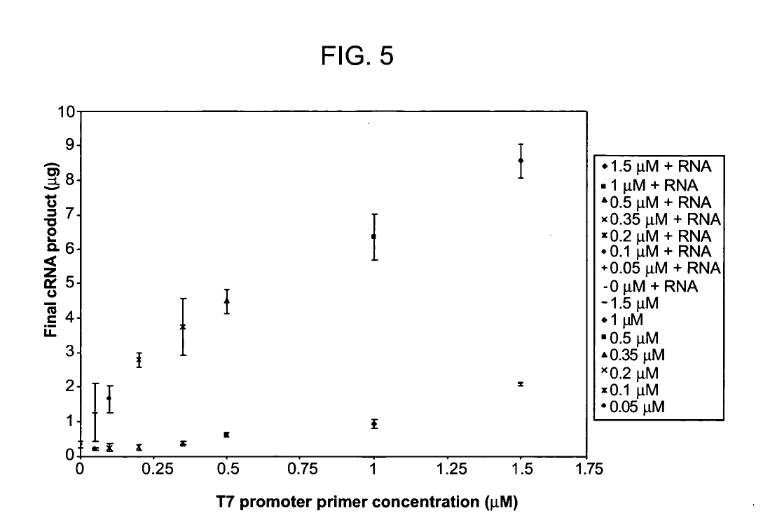

A feature of the present invention is that the amount of promoter-primer employed in the first reverse transcription step is chosen to provide for a high yield of selective RNA product in the second in vitro transcription step, as described in greater detail below. Specifically, the ratio amount of promoter-primer to initial RNA sample is selected so that a high yield of target specific RNA product (cRNA which is antisense RNA with respect to the initial mRNA) is produced by the in vitro transcription step. In many embodiments, the ratio is one that, in the absence of RNA sample, yields an amount of cRNA that is at or close to baseline of the UV measurement for cRNA yield, using the protocol described in the Experimental Section below in connection with FIG. 5. In other words, the amount ratio chosen is one that produces substantially no cRNA product in the absence of input RNA, where by substantially no cRNA product is meant that the amount detected, if any, is at or close to baseline of the UV measurement for cRNA yield, as described above, where close to baseline means that it deviates from baseline by no more than about 1.0 μg, such as no more than about 0.5 μg. In certain representative embodiments, the ratio amount of primer-promoter to input RNA ranges from about 1-10 pmol primer-promoter to about 1-500 ng total RNA.

The promoter-primer is contacted with the mRNA of the initial RNA sample under conditions that allow the primer domain, e.g., poly-dT site, to hybridize to the complementary region of the target mRNA, e.g., the poly-A tail present on most mRNA species in the total RNA sample. The resultant duplexes are then maintained under conditions sufficient to produce at least single stranded DNA, and in certain representative embodiments, double-stranded cDNA, from the duplexes. As such, the resultant duplexes are maintained in the presence of reagents necessary to, and for a period of time sufficient to, convert the primer-mRNA hybrids to at least single stranded, if not double stranded, cDNAs, depending on the nature of the desired transcription template product.

The catalytic activities required to convert primer-mRNA hybrid to singled stranded cDNA are an RNA-dependant DNA polymerase accitivity and a RNase H activity, while the catalytic activities required to convert primer-mRNA hybrid to double-stranded cDNA are an RNA-dependent DNA polymerase activity, a RNaseH activity, and a DNA-dependent DNA polymerase activity. Most reverse transcriptases, including those derived from Moloney murine leukemia virus (MMLV-RT), avian myeloblastosis virus (AMV-RT), bovine leukemia virus (BLV-RT), Rous sarcoma virus (RSV) and human immunodeficiency virus (HIV-RT) catalyze each of these activities. These reverse transcriptases are sufficient to convert primer-mRNA hybrid to double-stranded DNA in the presence of additional reagents which include, but are not limited to: dNTPs; monovalent and divalent cations, e.g. KCl, MgCl₂; sulfhydryl reagents, e.g. dithiothreitol; and buffering agents, e.g. Tris-Cl. Alternatively, a variety of proteins that catalyze one or two of these activities can be added to the cDNA synthesis reaction. For example, MMLV reverse transcriptase lacking RNaseH activity (described in U.S. Pat. No. 5,405,776) which catalyzes RNA-dependent DNA polymerase activity and DNA-dependent DNA polymerase activity, can be added with a source of RNaseH activity, such as the RNaseH purified from cellular sources, including Escherichia coli. These proteins may be added together during a single reaction step, or added sequentially during two or more substeps. Finally, additional proteins that may enhance the yield of double-stranded DNA products may also be added to the cDNA synthesis reaction. These proteins include a variety of DNA polymerases (such as those derived from E. coli, thermophilic bacteria, archaebacteria, phage, yeasts, Neurosporas, Drosophilas, primates and rodents), and DNA Ligases (such as those derived from phage or cellular sources, including T4 DNA Ligase and E. coli DNA Ligase).

In certain embodiments, a low molecular weight saccharide is included in the reaction mixture of the reverse transcription step, such that reverse transcription is carried out in the presence of a low molecular weight saccharide. Low molecular weight saccharides (i.e., sugars) of interest include monosaccharides, disaccharides and trisaccharides, where specific representative saccharides include, but are not limited to: trehalose, sucrose, raffinose, and the like. When present, the amount of saccharide will typically range from about 0.05M to about 2 M, usually from about 0.1 M to about 1 M and more usually from about 0.2 M to about 0.6 M.

Another component that is present in the reverse transcription reaction mixture in certain embodiments of the subject methods is a glycine based osmolyte. Glycine-based osmolytes suitable for use in the present invention include trimethylglycine (BETAINE™), glycine, sarcosine and dimethylglycine. Glycine based osmolytes are further described in U.S. Pat. No. 5,545,539, the disclosure of which is herein incorporated by reference.

Conversion of primer-mRNA hybrid to a transcription template product, e.g., double-stranded cDNA, by reverse transcriptase proceeds through an RNA:DNA intermediate which is formed by extension of the hybridized promoter-primer by the RNA-dependent DNA polymerase activity of reverse transcriptase. The RNaseH activity of the reverse transcriptase then hydrolyzes at least a portion of the RNA:DNA hybrid, leaving behind RNA fragments that can serve as primers for second strand synthesis (Meyers et al., Proc. Nat'l Acad. Sci. USA (1980) 77: 1316 and Olsen & Watson, Biochem. Biophys. Res. Commun. (1980) 97: 1376). Extension of these primers by the DNA-dependent DNA polymerase activity of reverse transcriptase results in the synthesis of double-stranded cDNA. Other mechanisms for priming of second strand synthesis may also occur, including “self-priming” by a hairpin loop formed at the 3′ terminus of the first strand cDNA (Efstratiadis et al. (1976), Cell 7, 279; Higuchi et al. (1976), Proc. Natl, Acad, Sci USA 73, 3146; Maniatis et al. (1976), Cell 8, 163; and Rougeon and Mach (1976), Proc. Natl. Acad. Sci. USA 73, 3418; and “non-specific priming” by other DNA molecules in the reaction, i.e. the promoter-primer.

In those embodiments where the the transcription template includes a second strand cDNA, the second strand cDNA synthesis results in the production of a double-stranded promoter region. The second strand cDNA includes not only a sequence of nucleotide residues that comprise a DNA copy of the mRNA template, but also additional sequences at its 3′ end which are complementary to the promoter-primer used to prime first strand cDNA synthesis. The double-stranded promoter region serves as a recognition site and transcription initiation site for RNA polymerase, which uses the second strand cDNA as a template for multiple rounds of RNA synthesis during the next stage of the subject methods.

Depending on the particular protocol, the same or different DNA polymerases may be employed during the cDNA synthesis step. For example, a single reverse transcriptase, most preferably MMLV-RT, may be used as a source of all the requisite activities necessary to convert primer-mRNA hybrid to double-stranded cDNA. Alternatively, the polymerase employed in first strand cDNA synthesis may be different from that which is employed in second strand cDNA synthesis. Specifically, a reverse transcriptase lacking RNaseH activity (e.g. Superscript II™) may be combined with the primer-mRNA hybrid during a first substep for first strand synthesis. A source of RNaseH activity, such as E. coli RNaseH or MMLV-RT, may be added during a second substep to initiate second strand synthesis. In yet other embodiments, the requisite activities are provided by a plurality of distinct enzymes. The manner is which double-stranded cDNA is produced from the initial mRNA is not critical to certain embodiments of the invention. However, in certain embodiments one employs MMLV-RT, or a combination of Superscript II™ and MMLV-RT, or a combination of Superscript II™ and E. coli RNaseH, for cDNA synthesis as these embodiments yield certain desired results.

Typically, the incubation or reaction period for the above-described reverse transcription step lasts for a period of time that ranges from about 0.5 hours to about 12 hours hours, such as from about 2 hours to about 6 hours, including from about 4 to about 8 hours.

The next step of the subject methods is the preparation of antisense RNA from the transcription template product, e.g., the double-stranded cDNA product, prepared in the first reverse transcription step. During this step, the transcription template product, e.g., the double-stranded cDNA, produced in the first reverse transcription step is transcribed by RNA polymerase to yield antisense RNA, which is complementary to the initial mRNA target from which it is amplified.

Depending on the particular protocol employed, the subject methods may or may not include a step in which the transcription template products, e.g., double-stranded cDNAs, produced as described above are physically separated from the reverse transcriptase employed in the cDNA production step prior to the transcription step. As such, in certain embodiments, the cDNAs produced in the first step of the subject methods are separated from the reverse transcriptase employed in this first step prior to the second transcription step described in greater detail below. In these embodiments, any convenient separation protocol may be employed, including the phenol/chloroform extraction and ethanol precipitation (or dialysis), protocol as described in U.S. Pat. Nos. 5,554,516 and U.S. Pat. No. 5,716,785, the disclosures of which are herein incorporated by reference.

In yet other embodiments, the subject methods do not involve a step in which the transcription template products, e.g., double-stranded cDNAs, are physically separated from the reverse transcriptase following double-stranded cDNA preparation. In these embodiments, the reverse transcriptase that is present during the transcription step is rendered inactive. Thus, the transcription step is carried out in the presence of a reverse transcriptase that is unable to catalyze RNA-dependent DNA polymerase activity, at least for the duration of the transcription step. As a result, the antisense RNA products of the transcription reaction cannot serve as substrates for additional rounds of amplification, and the amplification process cannot proceed exponentially.

The reverse transcriptase present during the transcription step may be rendered inactive using any convenient protocol, including those described in U.S. Pat. No. 6,132,997; the disclosure of which is herein incorporated by reference. As described in this reference, the transcriptase may be irreversibly or reversibly rendered inactive. Where the transcriptase is reversibly rendered inactive, the transcriptase is physically or chemically altered so as to no longer able to catalyze RNA-dependent DNA polymerase activity. The transcriptase may be irreversibly inactivated by any convenient means. Thus, the reverse transcriptase may be heat inactivated, in which the reaction mixture is subjected to heating to a temperature sufficient to inactivate the reverse transcriptase prior to commencement of the transcription step. In these embodiments, the temperature of the reaction mixture and therefore the reverse transcriptase present therein is typically raised to 55° C. to 70° C. for 5 to 60 minutes, usually to about 65° C. for 15 to 20 minutes. Alternatively, the reverse transcriptase is irreversibly inactivated by introducing a reagent into the reaction mixture that chemically alters the protein so that it no longer has RNA-dependent DNA polymerase activity. In yet other embodiments, the reverse transcriptase is reversibly inactivated. In these embodiments, the transcription may be carried out in the presence of an inhibitor of RNA-dependent DNA polymerase activity. Any convenient reverse transcriptase inhibitor may be employed which is capable of inhibiting RNA-dependent DNA polymerase activity a sufficient amount to provide for linear amplification. However, these inhibitors should not adversely affect RNA polymerase activity. Reverse transcriptase inhibitors of interest include ddNTPs, such as ddATP, ddCTP, ddGTP or ddTTP, or a combination thereof, the total concentration of the inhibitor typically ranges from about 50 μM to 200 μM.

Regardless of whether the cDNA is separated from the reverse transcriptase prior to the in vitro transcription step, for the in vitro transcription step, the presence of the RNA polymerase promoter region on the transcription template product, e.g., double-stranded cDNA, is exploited for the production of complementary RNA or cRNA, which is also properly viewed as antisense RNA with respect to its initial mRNA template. To synthesize the RNA, the double-stranded DNA is contacted with the appropriate RNA polymerase in the presence of the four ribonucleotides (i.e., UTP, ATP, GTP and CTP), under conditions sufficient for RNA transcription to occur, where the particular polymerase employed will be chosen based on the promoter region present in the double-stranded DNA, e.g. T7 RNA polymerase, T3 or SP6 RNA polymerases, E. coli RNA polymerase, and the like. Suitable conditions for RNA transcription using RNA polymerases are known in the art, see e.g. Milligan and Uhlenbeck (1989), Methods in Enzymol. 180, 51.

In certain embodiments, this cRNA synthesis step (which may be viewed as an in vitro transcription step) is carried out the presence of a diol. Diols of interest are generally liquids at physiologic temperatures and include diols of from about 8 to about 28 carbon atoms, such as from about 16 to about 20 carbon atoms, where the diol may be a polyoxyalkylene diol, where alkylene is from about 2 to about 3 carbon atoms, however, diols having significantly more carbon atoms also find use. Suitable diols for use as cosolvents may range from about 200 to about 10,000 daltons, such as from about 300 to about 10,000 daltons, including from 400 to about 10,000 daltons, e.g., from about 800 to about 10,000 daltons. Diols of particular interest include polyethylene glycols, particularly polyethylene glycol 200 (PEG₂₀₀), polyethylene glycol 300 (PEG₃₀₀), polyethylene glycol 400 (PEG 400), polyethylene glycol 600 (PEG 600), polyethylene glycol 1000 (PEG₁₀₀₀), polyethylene glycol 3400 (PEG 3400), polyethylene glycol 8000 (PEG₈₀₀₀), and the like. When present in the in vitro transcription reaction mixture, the diols will usually be present in an amount ranging from about 1% to about 10%, such as from about 2% to about 8%, including from about 4% to about 6%.

In certain embodiments, the RNA products of the above described in vitro transcription step are labeled. In these embodiments, the reagents employed in the subject transcription reactions typically include a labeling reagent, where the labeling reagent may be a directly or indirectly detectable label. A directly detectable label is one that can be directly detected without the use of additional reagents, while an indirectly detectable label is one that is detectable by employing one or more additional reagent, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g. fluorescently tagged CTP (such as Cy3-CTP, Cy5-CTP) etc. Fluorescent moieties which may be used to tag nucleotides for producing labeled probe nucleic acids include, but are not limited to: fluorescein, the cyanine dyes, such as Cy3, Cy5, Alexa 555, Bodipy 630/650, and the like. Other labels may also be employed as are known in the art.

The above protocol results in the production of an amplified population of cRNA nucleic acids that are antisense with respect to the mRNAs employed in the dsDNA reverse transcription step. In certain embodiments, the product RNA nucleic acids are labeled, as described above. Where desired, the resultant RNA product nucleic acids may be separated from the remainder of the reaction mixture, where any convenient separation protocol may be employed.

Utility

The resultant antisense RNA produced by the subject methods finds use in a variety of applications. For example, the resultant antisense RNA can be used in expression profiling analysis on such platforms as DNA microarrays, for construction of “driver” for subtractive hybridization assays, for cDNA library construction, and the like. Especially facilitated by the subject methods are studies of differential gene expression in mammalian cells or cell populations. The cells may be from blood (e.g., white cells, such as T or B cells) or from tissue derived from solid organs, such as brain, spleen, bone, heart, vascular, lung, kidney, liver, pituitary, endocrine glands, lymph node, dispersed primary cells, tumor cells, or the like. The RNA amplification technology can also be applied to improve methods of detecting and isolating nucleic acid sequences that vary in abundance among different populations using the technique known as subtractive hybridization. In such assays, two nucleic acid populations, one sense and the other antisense, are allowed to mix with one another with one population being present in molar excess (“driver”). Under appropriate conditions, the sequences represented in both populations form hybrids, whereas sequences present in only one population remains single-stranded. Thereafter, various well-known techniques are used to separate the unhybridized molecules representing differentially expressed sequences. The amplification technology described herein may be used to construct large amounts of antisense RNA for use as “driver” in such experiments.

Depending on the particular intended use of the subject antisense RNA, the antisense RNA may be labeled. One way of labeling which may find use in the subject invention is isotopic labeling, in which one or more of the nucleotides is labeled with a radioactive label, such as ³²S, ³²P, ³H, or the like. Another means of labeling is fluorescent labeling in which fluorescently tagged nucleotides, e.g. CTP, are incorporated into the antisense RNA product during the transcription step. Fluorescent moieties which may be used to tag nucleotides for producing labeled antisense RNA include: fluorescein, the cyanine dyes, such as Cy3, Cy5, Alexa 555, Bodipy 630/650, and the like. Other labels may also be employed as are known in the art.

In certain embodiments, the antisense RNA produced by the subject methods is employed as template in the preparation of labeled deoxyribonucleic acid molecules, e.g., labeled target DNA molecules. To prepare labeled target DNA molecules from the antisense or cRNA product of the subject methods, cRNA product is typically contacted with a suitable primer, catalytic activities and other reagents required to generate labeled target nucleic acid from the RNA template molecules. The primers may be any of a number of different kinds of primers known to those of skill in the art, including a random hexamer primers, gene specific primers, etc. The catalytic activities employed typically include an RNA-dependent DNA polymerase activity, i.e., a reverse transcriptase, which may or may not have RNase H activity, where representative reverse transcriptases are discussed above. In such, methods, the (+) strand RNA templates are contacted with the reverse transcriptase and other reagents, where the additional reagents may include, but are not limited to: dNTPs; labeled dNTPs, monovalent and divalent cations, e.g. KCl, MgCl₂; sulfhydryl reagents, e.g. dithiothreitol; and buffering agents, e.g. Tris-Cl; under conditions sufficient to produce the desired labeled target deoxyribonucleic acids, where such conditions are well known to those of skill in the art.

As such, the subject methods of nucleic acid generation find use in nucleic acid analyte detection applications, where the subject methods are employed to generate the nucleic acid analyte. Specific analyte detection applications of interest include hybridization assays in which the nucleic acid produced by the subject methods are hybridized to arrays of probe nucleic acids.

An “array”, unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

In these assays, a sample of target nucleic acids is first prepared according to the methods described above, where preparation may include labeling of the target nucleic acids with a label, e.g. a member of signal producing system. Following sample preparation, the sample is contacted with an array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. patent applications: Ser. No. 09/846,125 “Reading Multi-Featured Arrays” by Dorsel et al.; and U.S. Pat. No. 6,406,849, which references are incorporated herein by reference. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Kits

Also provided are kits for use in the subject invention, where such kits may include containers, each with one or more of the various reagents (typically in concentrated form) utilized in the methods, including, for example, buffers, the appropriate nucleotide triphosphates (e.g. dATP, dCTP, dGTP, dTTP, ATP, CTP, GTP and UTP), reverse transcriptase, RNA polymerase, promoter-primer, saccharide, glycine osmolyte, diol, etc. Also present in the kits may be total RNA isolation reagents, e.g., RNA extraction buffer, proteinase digestion buffer; proteinase K, etc.

Finally, the kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

The following examples are offered by way of illustration and not by way of limitation.

### Experimental

I. Comparison of RNA Yields from Standard and Low Input Protocols

A. Low Input Protocol

The Low Input protocol is listed in section VIB, below.

B. Standard Protocol

The Standard Protocol is similar to the Low Input protocol, with the following changes:
5× First Strand buffer does not contain Raffinose or Triton X-100
30 pmol of T7 primer were added compared to 7.2 pmol of T7 primer used in the Low Input protocol
Transcription reaction did not contain PEG8000.
   C. Results

RNA was amplified from various input amounts of total RNA from different cell types using either the standard or low input protocol described above. The results are provided in Table 1, below.

| | | Standard | |
|---|---|---|---|
| Input total RNA | | Protocol | Low Input Protocol |
| amount (ng) | Cell Type | Yield (μg) | Yield (μg) |
| 50 | HeLa | 1.5 +/− 0.3 | 1.5 +/− 0.1 |
| 500 | | 5.6 +/− 0.4 | 10.3 +/− 0.4 |
| 5000 | | 34.3 +/− 3.7 | 39.7 +/− 1.1 |
| 200 ng PolyA+ | | 53.4 +/− 4.4 | 49.5 +/− 0.1 |
| 50 | Spleen | 1.7 +/− 0.2 | 1.0 +/− 0.1 |
| 500 | | 4.0 +/− 0.7 | 6.1 +/− 0.7 |
| 5000 | | 14.1 +/− 3.5 | 28.2 +/− 2.2 |
| 200 ng PolyA+ | | 44.0 +/− 1.6 | 47.2 +/− 0.9 |
| 50 | Heart | 2.2 +/− 0.3 | 1.5 +/− 0.1 |
| 500 | | 4.3 +/− 0.2 | 7.0 +/− 0.7 |
| 5000 | | 18.8 +/− 7.3 | 30.4 +/− 9.0 |
| 200 ng PolyA+ | | 36.1 +/− 1.3 | 67.6 +/− 1.2 |
| 50 | Liver | 1.5 +/− 0.2 | 1.4 +/− 0.2 |
| 500 | | 4.5 +/− 0.7 | 7.0 +/− 1.2 |
| 5000 | | 24.2 +/− 3.9 | 32.4 +/− 4.8 |
| 200 ng PolyA+ | | 35.7 +/− 5.4 | 67.4 +/− 5.8 |

The Standard Protocol does yield cRNA from low amounts of RNA. However, hybridization of these samples to an Agilent Human 1A oligo (G4140A) array (Agilent Technologies, Palo Alto Calif.) under according to the manufacturer's instructions (see user's manual (G4140-90050) shows that the cRNA generated is not necessarily made up of specific targets that bind specifically to the probes in the features on the microarray. Instead, the product may be a combination of amplified targets and products resulting from the non-specific transcriptional activity of T7 RNA polymerase.
II. Linear Correlation of the Log Ratio of Amplified cRNA as a Function of Sample Input
A. Methods

1 μg of cy3-labeled cRNA generated from 50-2500 ng Hela total RNA was cohybridized with 1 μg cy5-labeled cRNA generated from 50-2500 ng Spleen total RNA onto an Agilent human 1A oligo microarray (Agilent Technoligies, Palo Alto Calif.). The microarray was hybridized, washed, and scanned on the Agilent Microarray Scanner according to the manufacturer's instructions as described in the user's manual (G4140-90550). Each microarray was feature extracted and linear correlations of the log ratios on a feature by feature basis as function of RNA sample input were determined.

B. Results

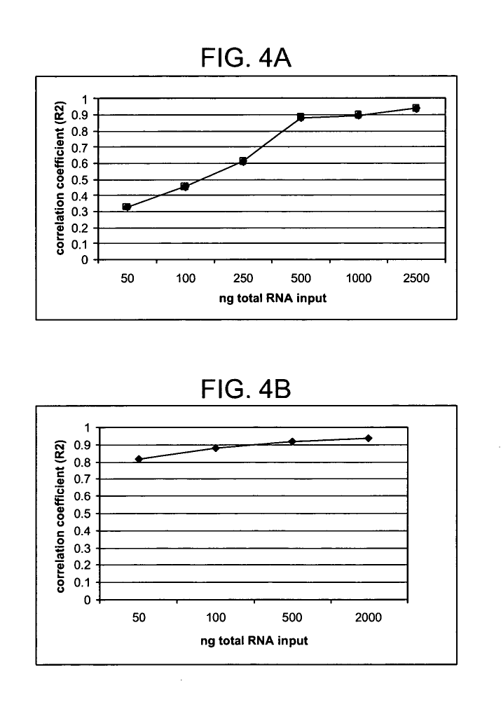

FIG. 4A is the Standard protocol, showing the Log Ratio correlation between 5000 ng and decreasing amounts of total RNA input. FIG. 4B is the Low Input protocol, showing the correlation between 200 ng and decreasing amounts of total RNA input. The correlation coefficient drops significantly when the RNA input is less than 500 ng when using the Standard protocol. In contrast, the Low Input protocol shows strong correlation at each RNA sample input amount.

III. Titration Curve of T7 Promoter Primer from 0.05 μM to 1.5 μM in the Presence and Absence of 100 ng of HeLa total RNA.

cRNA was amplified with various amounts of T7 promoter primer and a constant amount of input total RNA using the low input protocol described above. The results are provided in FIG. 5. At high concentrations of T7 promoter primer alone (1.5 μM) in the RT reaction, amplified yield is 1.58 μg. As the concentration of the primer decreases, this spurrious yield also decreases. The primer concentration should be brought down to a point when this spurrious yield is minimal, and it shows to be at 0.35 μM from the graph.

IV. Comparison of the Effect of Two Additives to the RT Reaction with 100 ng of HeLa Total RNA.

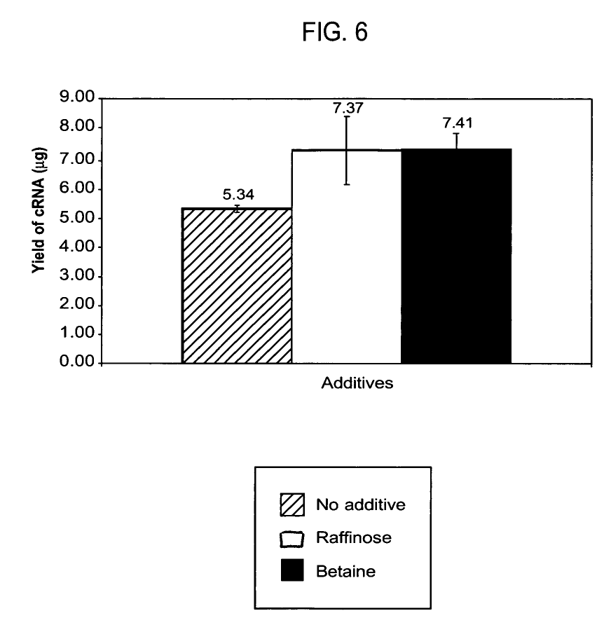

The effects of either raffinose or betaine when included in the RT reaction substep of the low input protocol described above were evaluated. As shown in FIG. 6. There is 34% of improved yield in the presence of raffinose or betaine.

V. Comparison of Results Obtained by Standard Protocol on Various Amounts of Input RNA

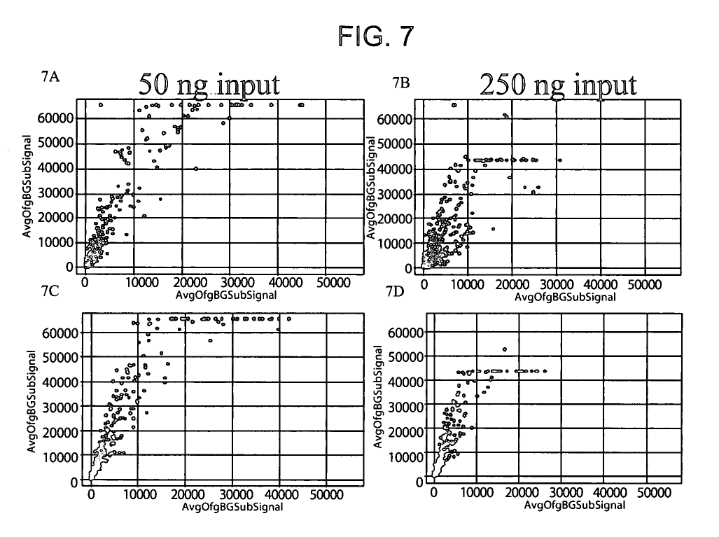

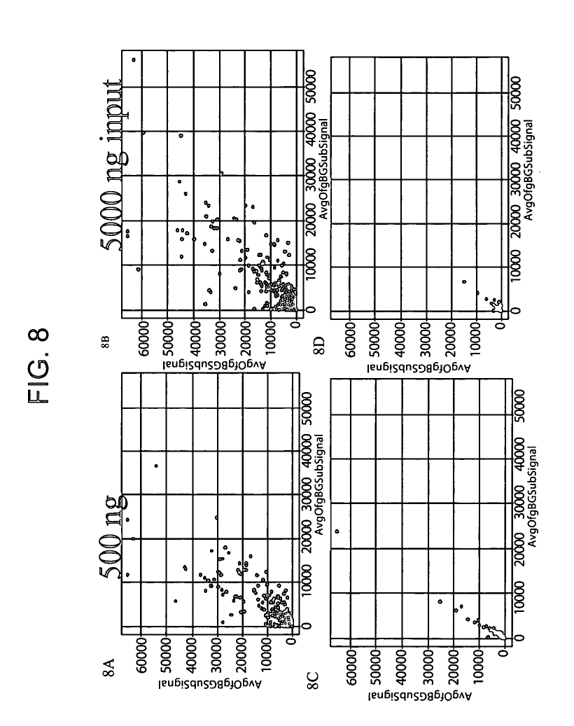

The Standard protocol as described above was used to generate fluroescently-labeled cRNA. The targets were hybridized to an array that contains probes that bind to the targets (binding probes), and probes that are the reverse complement of the binding probes (nonbinding probes) using the protocol described in the Agilent oligo microarray user's manual. FIGS. 7A, 7B, 8A and 8B under each total RNA input amount show labeled cRNA binding to the gene specific probes and FIGS. 7C, 7D, 8C and 8D show binding to the reverse complement or nonbinding probes. Consistent with the above data, when the sample RNA input is less than 500 ng, T7 nonspecific targets are generated and bind to the nonbinding probes on the microarray, resulting in high signal intensities. When the sample RNA input is above 500 ng, the amount of T7 nonspecific targets is significantly reduced, as shown by the large decrease in the signal intensities of the nonbinding probes.

VI. Representative Protocols

A. Reaction Components

T7 Promoter Primer (5′) AAT TAA TAC GAC TCA CTA TAG GGA GAT TTT TTT TTT TTT TTT TTV N(3′) (V=A/CIG, N=A/C/G/T) (6 μM) (SEQ ID NO:01)
5× First Strand Buffer (250 mM Tris-HCl, pH8.3, 15 mM MgCl₂, 375 mM KCl, 1 M Raffinose, 0.075% (v/v) Triton X-100)
DTT(0.1 M)
dNTPs (A) (10 mM dATP, dCTP, dGTP, dTTP)
MMLV-RT (200 U/μl)
RNase OUT (40 U/μl)
4× Transcription Buffer (160 mM Tris-HCl, pH 8.0, 55 mM MgCl₂, 100 mM NaCl, 8 mM spermidine)
NTPs (25 mM ATP, GTP, UTP, 7.5 mM CTP)
Inorganic Pyrophosphatase (200 U/ml)
T7 RNA Polymerase (2500 U/μl)
PEG 8000 (50% (w/v)
Cy3-CTP (10 mM)
Cy5-CTP (10 mM)
   B. Protocol for Amplification of Low Input Total RNA to Generate Fluorescently Labeled-cRNA Target for Hybridization onto Oligo Microarray for Six Reactions
   1. First cDNA Synthesis Reaction
   Add 5-1,000 ng total RNA to reaction tube.
   Add 1.2 μl T7 Promoter Primer (7.2 pmol) and bring total sample volume to 11.5 μl in nuclease-free water.
   Incubate sample at 65° C. for 10 min to denature primer and template. Keep the reaction tube on ice for 5 min.
   2. cDNA Mix Method

Pre-warm 5× First Strand Buffer in 65° C. waterbath for 3-4 minutes. Vortex the vial for 5s and spin it on the benchtop centrifuge for 5 seconds. Mix the following components and maintain at room temp.

Preparation of 1^{st} cDNA Mix

| | Volume (μl)/ | Volume (μl)/ |
|---|---|---|
| Component | reaction | 6.5 reactions |
| 5x First Strand Buffer | 4.0 | 26 |
| 0.1 M DTT | 2.0 | 13 |
| 10 mM dNTP Mix (A) | 1.0 | 6.5 |
| MMLV-RT | 1.0 | 6.5 |
| RNaseOUT | 0.5 | 3.3 |
| Volume of cDNA Mix | 8.5 | 55.3 |

Aliquot 8.5 μl of cDNA Mix into each sample tube.
Incubate cDNA synthesis reaction at 40° C. for 120 min. (2 hours)
Incubate reaction tube at 65° C. for 15 min.
Keep the reaction tube on ice for 5 minutes before moving to transcription step.
3. Transcription Reaction

Immediately before use, mix the following components in the order indicated at room temperature.

Preparation of Transcription Mix

| | Volume | Volume (μl)/6.5 |
|---|---|---|
| Component | (μl)/reaction | reactions |
| Nuclease-free water | 12.1 | 78.65 |
| 4x Transcription Buffer | 20 | 130 |
| 0.1 M DTT | 6.0 | 39 |
| NTP Mix | 8.0 | 52 |
| 100 mM CTP | 5.6 | 36.4 |
| RNaseOUT | 0.5 | 3.3 |
| Inorganic Pyrophosphatase | 0.6 | 3.9 |
| T7 RNA polymerase | 0.8 | 5.2 |
| 50% PEG 8000 pre-warmed in 40 C. | 6.4 | 41.6 |
| water bath for 1 minute | | |
| Volume of Transcription Mix | 60 | 390 |

Aliquot 60 μl of Transcription Mix into each sample tube.
Incubate transcription reactions at 40° C. for 120 min (2 hours).
Purify cRNA target using Qiagen RNeasy Column.
Speed vac the sample until dry.

The above results and discussion demonstrate that novel and improved methods of producing linearly amplified amounts of antisense RNA from an initially small mRNA source, e.g., total RNA, are provided. The subject methods provide for an improvement over prior methods of producing antisense RNA in that the one can use very small amounts of input RNA and obtain sufficient yields of highly specific cRNA therefrom, where the product cRNA is highly representative of the mRNA population of the initial RNA sample. As such, the subject methods represent a significant contribution to the art.

All publications and patent application cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for producing linearly amplified amounts of cRNA from an initial RNA sample, said method comprising:
(a) reverse transcribing said initial RNA sample into a transcription template product comprising at least a single-stranded cDNA domain and a double stranded RNA polymerase promoter domain by contacting said initial RNA sample with a promoter-primer under conditions sufficient to produce said transcription template product, wherein said initial RNA sample and said promoter-primer are contacted in an amount ratio that produces substantially no product cRNA in the absence of input RNA; and
(b) transcribing said transcription template product into cRNA.

2. The method according to claim 1, wherein said transcription template product is produced by reverse transcribing said initial RNA sample into double-stranded cDNA having a terminal RNA polymerase-promoter region from by:
(i) contacting said initial RNA sample with said promoter-primer under conditions wherein mRNA present in said initial RNA sample forms a complex with said promoter-primer, wherein said promoter-primer comprises an mRNA binding site linked to a promoter sequence; and
(ii) converting said complex to double-stranded cDNA using a combination of RNA-dependent DNA polymerase activity, RNaseH activity and DNA-dependent DNA polymerase activity.

3. The method according to claim 1, wherein the amount ratio of said initial RNA sample to said primer-promoter ranges from about 1-500 ng initial RNA to 1-10 pmol primer-promoter.

4. The method according to claim 1, wherein said initial RNA sample is total RNA.

5. The method according to claim 1, wherein said reverse transcribing step occurs in the presence of a thermostabilizing agent.

6. The method according to claim 3, wherein said thermostabilizing agent is a sugar.

7. The method according to claim 1, wherein said reverse transcribing step occurs in the presence of a glycine based osmolyte.

8. The method according to claim 1, wherein said transcribing step occurs in the presence of a polyalkylene oxide.

9. The method according to claim 9, wherein said polyalkylene oxide is a polyethylene glycol.

10. The method according to claim 1, wherein the amount of said initial RNA sample employed in said producing step (a) does not exceed about 500 ng.

11. A method for producing linearly amplified amounts of cRNA from an initial RNA sample, said method comprising:
(a) reverse transcribing double-stranded cDNA having an RNA polymerase-promoter region from said initial RNA sample not exceeding about 500 ng by:
(i) contacting said initial RNA sample with a promoter-primer under conditions wherein mRNA present in said initial RNA sample forms a complex with said promoter-primer, wherein said promoter-primer comprises an mRNA binding site linked to a promoter sequence; and
(ii) converting said complex to double-stranded cDNA using a combination of RNA-dependent DNA polymerase activity, RNaseH activity and DNA-dependent DNA polymerase activity,
wherein said initial RNA sample and said primer-promoter are contacted in an amount ratio that produces a high yield of target specific cRNA product in said transcribing step (b); and
(b) transcribing said double-stranded cDNA having a terminal RNA polymerase-promoter region into cRNA.

12. The method according to claim 11, wherein the amount ratio of said initial RNA sample to said primer-promoter ranges from about 1-500 ng initial RNA to 1-10 pmol primer-promoter.

13. The method according to claim 11, wherein said initial RNA sample is total RNA.

14. The method according to claim 11, wherein said reverse transcribing step occurs in the presence of a thermostabilizing agent.

15. The method according to claim 13, wherein said thermostabilizing agent is a sugar.

16. The method according to claim 1, wherein said reverse transcribing step occurs in the presence of a glycine based osmolyte.

17. The method according to claim 11, wherein said transcribing step occurs in the presence of a polyalkylene oxide.

18. A kit for use in linearly amplifying mRNA into cRNA, said kit comprising:
an oligonucleotide promoter-primer comprising an RNA polymerase promoter sequence; and
instructions for practicing the method according to claim 1.

19. The kit according to claim 18, wherein said kit further comprises at least one thermostabilizing agent.

20. The kit according to claim 18, wherein said kit further comprises at least one glycine based osmolyte.

21. The kit according to claim 18, wherein said kit further comprises at least one polyalkylene oxide.

22. A method of detecting the presence of a nucleic acid analyte in a linearly amplified sample produced from an initial RNA sample according to claim 1, said method comprising:
(a) contacting said linearly amplified sample with a nucleic acid array;
(b) detecting any binding complexes on the surface of the said array to obtain binding complex data; and
(c) determining the presence of said nucleic acid analyte in said sample using said binding complex data.

23. A method comprising transmitting data representing a result obtained by the method according to claim 22, from a first location to a second location.

24. A method according to claim 23, wherein said second location is a remote location.

25. A method comprising receiving data representing a result of a reading obtained by the method of claim 22.
